(19) United States
(12) Patent Application Publication
(10) Pub. No.: US 2003/0166594 A1
(43) Pub. Date: Sep. 4, 2003

(54) Nucleic acid delivery system

(76) Inventors: Holly G Blum, Manchester by the Sea, MA (US); Per Artursson, Uppsala (SE)

Correspondence Address: Patent Administrator, Testa Hurwitz & Thibeault, High Street Tower, 125 High Street, Boston, MA, 02110, US

(21) Appl. No.: 10/149,458

(22) PCT Filed: Dec. 7, 2000
(86) PCT No.: PCT/EP00/12339

(30) Foreign Application Priority Data
Dec. 8, 1999 (SE) 9904475.2

(51) Int. Cl. A61K 48/00; C12N 15/85; C12P 21/02; C12N 5/06

(52) U.S. Cl. 514/44; 435/455; 435/69.1; 435/320.1; 435/325

(57) ABSTRACT

The present invention is directed to a composition and pharmaceutical preparations for introducing nucleic acids including oligo- or poly-nucleotide into cells in a host tissue by a delivery system and a method of preparing such a composition. The composition for delivery of nucleic acids comprises polymeric carrier particles that are essentially free of groups having a positive electric charge and the nucleic acids are provided essentially on the surface of the particles. The carrier particle is insoluble in water but suitably it is able to absorb water quickly.

### FIELD OF THE INVENTION

The present invention relates to a non-viral, non-condensing delivery system for nucleic acids, and more specifically, to a system, which facilitates the introduction of nucleic acid into cells in a host tissue after administration to that tissue. This formulation is in the form of a stable and concentrated dry powder that efficiently delivers biologically active nucleic acids such as oligo- or poly-nucleotides in a non-condensed form to a host tissue, such as a mucosal tissue, and facilitates the expression of a desired product or activity in cells present in that tissue.

### BACKGROUND ART

Nucleic acids have been applied to gene therapy, antisense therapy and in genetic vaccination and have many advantages over traditional medicines/vaccines including proteins and peptides. However, from a pharmaceutical point of view, the way of delivery of nucleic acids still remains a challenge since a relatively low expression is obtained in vivo as compared to viral-based gene delivery systems (Saeki et al. 1997).

The negatively charged nucleic acids such as oligo- or poly-nucleotides are most commonly delivered after complexation and condensation with positively charged molecules such as cationic lipids, cationic peptides or cationic polymers (Felgner et al. 1987; Gao et al. 1995; Kabanov et al. 1995). These cationic molecules may provide protection against nuclease degradation and may also give the nucleic acid-cationic molecule complex surface properties that favour their interaction with and uptake by the cells. For instance, cationic lipids have been extensively studied and shown to mediate efficient expression of a variety of genes in many in vitro systems and also in some in vivo systems (Feigner et al. 1987; Deshpande et al. 1998; Freimark et al. 1998; Guillaume-Gable et al. 1998; Pillai et al. 1998; Sternberg et al. 1998).

Interestingly, while these delivery systems are efficient in some tissues, mainly the lung tissues (Deshpande et al. 1998; Freimark et al. 1998; Guillaume-Gable et al. 1998; Pillai et al. 1998; Sternberg et al. 1998), they remain inefficient in other tissues. Thus, gene transfection in vivo is tissue-dependent in an unpredictable way and therefore remains a challenge.

For instance, formulations of cationic lipid and plasmid DNA (pDNA) optimized for gene expression in murine lung did not facilitate gene expression in the nasal epithelium as compared with naked pDNA solutions and only a marginal correction of the ion transport defects in cystic fibrosis transgenic mice was observed (Jiang et al. 1998). It has been shown that naked pDNA transfected the nasal epithelium cells more efficiently than condensed complexes using the polycationic polyethylenimine (PEI) or chitosan (Köping-Höggård et al. 1998). Moreover, cationic complex formulations have been shown to be inefficient as gene delivery system not only in tissues like nasal epithelium, but also in certain other tissues. Injection of naked pDNA into mouse brain or muscle resulted in dose-dependent gene expression that was not improved by complexation with cationic lipids (Schwartz et al. 1996).

In contrast, delivery systems comprised of pDNA and anionic lipids enhanced transgene expression significantly in mouse brain, muscle and liver, whereas the use of cationic lipids dramatically reduced gene expression (Schwartz et al. 1995; Saeki et al. 1997).

However, many other studies have observed that in general, anionic or neutral lipids are less effective or ineffective as compared to cationic lipids in terms of in vitro and in vivo gene transfection (Barthel et al. 1993; Fasbender et al. 1995; Lee et al. 1997).

Other studies, including one clinical trial on gene transfer to the airway epithelia in cystic fibrosis patients, showed that a naked pDNA solution was at least as effective as cationic liposome pDNA complexes (Meyer et al. 1995; Zabner et al. 1997).

Thus, it is difficult to draw any general conclusions regarding the gene transfection efficiency of cationic complexes formulations of pDNA in various tissues.

However, the approach of administration of naked pDNA has been considered safe enough for immunization of infants (Bot et al. 1998) and the pDNA constructs can simply be injected at a desired site, normally to a muscle (Armstrong et al. 1997). Thus, the use of non-condensed pDNA, e.g. in the form of naked pDNA solutions, may be an interesting means for gene transfer to certain tissues.

Since the first report on gene expression after intramuscular administration of a naked pDNA solution to mice (Wolff et al. 1990), only a few efforts to improve the delivery of the non-condensed pDNA have been made.

Injection of pDNA solutions complexed with non-condensing but ‘interactive’ polymers such as polyvinyl pyrrolidone (PVP) and polyvinyl alcohol (PVA) has resulted in about 10-fold enhancement of gene expression over naked pDNA control in rat muscle (Mumper et al. 1996; Mumper et al. 1998; Barry et al. 1999). An excess of these soluble polymeric molecules were found to interact with and stabilize the pDNA molecules in solution.

Another approach used materials with slow release properties such as poly-lactic-co-glycolic acid (PLGA) molecules. PLGA microspheres of relatively small size (a diameter of less than 10 μm) have been found suitable for cellular uptake.

In this particulate system, the nucleic acid is entrapped in the polymeric network of the carrier by certain preparation techniques (Hedley et al. 1998; Hedley et al. 1998; Lewis et al. 1998; Ando et al. 1999). For example, the nucleic acid and the polymer may be dissolved in various liquid phases, followed by an emulsification step and formation of polymeric particles by a coacervation process resulting in micro- or nano-particles wherein the nucleic acid is relatively homogeneously incorporated into the polymeric matrix.

Characteristic of such particulate formulations is that they deliver pDNA in a sustained and controlled way that is related to the degradation of the polymeric particles. However, such particulate formulations are rather complex and also expensive to prepare. Furthermore, they are not suitable to be used for immediate release of the pDNA directly after contact with the target cell, e.g. in a mucosal environment, since the release of pDNA is dependent on the degradation of the polymer matrix (Capan et al. 1999; Wang et al. 1999). Finally, to be efficient, such particle formulation may require that the target cell is capable of phagocytosing the particles. It is generally accepted that to be phagocytosed, a particle has to be less than 10 μm in diameter (Miglierina 1986). Thus, it is likely that such particles are not efficient in introducing nucleic acid into cells that have no, or only a low, phagocytosis capacity, such as epithelial cells lining mucosal tissues.

### SUMMARY OF THE INVENTION

The present invention is directed to a composition for delivery of nucleic acid into cells in a host tissue, preferably cells of a mucosal tissue, and also to a method for cheap and convenient preparation of such a composition. The basis for the present invention is the unexpected result that a large particulate carrier comprised of at least one polymer, such as cellulose, that is essentially free of groups having a positive charge and carries nucleic acid, such as pDNA, essentially on its surface can be used to deliver the nucleic acid into cells of a selected tissue. Surprisingly, the nucleic acid molecules that are rapidly released from surfaces of the insoluble polymeric particles, are taken up by the target cells and the desired molecules encoded for by the various nucleic acids are efficiently expressed in these cells. This formulation of pDNA is cheap and easy to prepare to a stable, e.g. a dry, formulation. It should therefore be easy to scale up with regard to production.

More specifically, the present invention is directed to a composition and to pharmaceutical preparations for introducing nucleic acids such as oligo- or poly-nucleotides into a host tissue by an insoluble polymeric delivery system. The polymeric carrier particle is comprised of one or more polymers that are essentially insoluble in extracellular body fluids such as mucosal fluids and are essentially free of chemical groups with positive charges. The invention provides methods for preparing such a delivery system, which system is suitable for administration of nucleic acids including oligo- or poly-nucleotides to a mammal.

In most cases, the negatively charged nucleic acids such as oligo- or poly-nucleotides are delivered into a host tissue after complex formation with positively charged molecules such as cationic lipids, cationic peptides or cationic polymers (Felgner et al. 1987; Gao and Huang 1995; Kabanov and Kabanov 1995). The present invention, on the other hand, is dealing with insoluble carrier molecules that are essentially free of chemical groups having a positive charge and/or are essentially electrically neutral rather than essentially cationic.

More specifically, previous formulations of DNA complexes using cationic lipids, cationic peptides or cationic polymers use the cation groups to neutralize the negative charges on the phosphates or analogous backbone groups of DNA, RNA, or another nucleic acid. In contrast, the carrier particles of the present invention do not have immobilized positive charges in sufficient density that the immobilized positive charges can neutralize the negative charges on the nucleic acid. Thus, when the DNA/carrier particle compositions of the invention are formulated, the negatively charged backbone groups generally must be neutralized by cations derived from a solution and not the carrier particle. For example, in a preferred embodiment, the carrier particles have a low enough density of immobilized cationic groups that less than 10 percent of the negative charges on the DNA can be effectively neutralized. In a more preferred embodiment, the carrier particles have a low enough density of immobilized cationic groups that less than 1 percent of the negative charges on the DNA can be effectively neutralized.

The basis for the composition of the present invention is the use of preformed carrier particles (without nucleic acid) onto which nucleic acid is provided or attached.

This is in contrast to previously known techniques, where the particles of the drug delivery system are formed from a liquid solution where both the nucleic acid and the polymers are molecularly dispersed. Such formation of nucleic acid-containing particles has often been prepared by the use of emulsification techniques or by coacervation procedures (Jones et al. 1997; Jones et al. 1997; Hedley et al. 1998; Lewis et al. 1998; Ando et al. 1999; Shea et al. 1999).

Previously used preparation techniques result in an important difference in the location of the nucleic acid as compared to the present invention. In prior literature, the nucleic acid is incorporated within a network of a polymeric matrix (Lewis et al. 1998; Shea et al. 1999), e.g. the nucleic acid is distributed throughout the interior of the particles. Alternatively, the nucleic acid is encapsulated by a polymeric shell. These types of entrapment procedures generally result in a slow-release of the entrapped compounds. A general characteristic of such slow-release formulations is that the encapsulating matrix will dissolve to release the nucleic acid. In contrast, according to the nucleic acid/carrier particle formulations of the present invention, the carrier particles are relatively insoluble and much of the nucleic acid is released from the carrier on a time scale that is much faster than the solubilization, degradation, or physical destruction of the carrier particle.

In contrast, according to the present invention the nucleic acid is provided or attached essentially at the surfaces of the carrier particles e.g. by simply soaking the carrier particles in a nucleic acid solution. Hereby the nucleic acid is preferentially found on the peripheral surfaces of the particles and when the particles are comprised of particle aggegates, also on the surfaces of the pores that extend from the peripheral surface into the interior of the carrier particles.

The method of preparing the composition comprises providing or attaching the selected nucleic acids onto the present polymeric carriers with an addition of one or several stabilizing agents such as a cryoprotectant, drying the mixture into a dry cake and milling of the dry cake into dry powders. The dry powder composition contains concentrated nucleic acids provided essentially on the surfaces of the carrier particles. The carrier particles are essentially insoluble in water but according to a suitable embodiment they absorb water.

The present invention further relates to pharmaceutical preparations based on this delivery system for preventative or therapeutic purposes, such as genetic vaccination or antisense and gene therapy, especially in tissues where ‘naked’ or non-condensed nucleic acids transfect more efficiently than condensed nucleic acids. Further, the present invention specifically relates to rapid release of the nucleic acid and subsequent cell transfection.

More specifically, the present invention is directed to a composition as defined in claim 1. Further embodiments of the invention are directed to the subject matter of claims 2-47.

### BRIEF DESCRIPTION OF THE DRAWINGS

FIG. 1 is a scanning electron micrograph that shows the general appearance of the cellulose carrier particles. The scale bar indicates 200 μm. The cellulose carrier particles were produced as described in Example 1.

FIG. 2 is a scanning electron micrograph that shows a closer view of a typical cellulose carrier particle. It is shown here that the cellulose carrier particle aggregate has a rough and very porous surface, with large pores that extends into the interior of the particles. The scale bar indicates 20 μm.

FIG. 3 shows an example of size analysis of a batch of the cellulose carrier particles determined by a Coulter counter particle size analyzer. In this case, the mean diameter is 29.30 μm. The particles seem to be essentially normally distributed with diameters ranging from 10 to 60 μm.

FIGS. 4a and 4b show a composition of cellulose carrier particles and fluorescently labeled pDNA as pictured by a confocal laser-scanning microscope. Cellulose carrier particles free of the fluorescently labeled pDNA served as control (FIG. 4c). The scale bars in (a), (b) and (c) represent a distance of 100, 50 and 20 μm, respectively.

FIG. 5 shows confocal optical sections from top to bottom of a composition of cellulose carrier particles and fluorescently labelled pDNA, (a) represents the first top section while (j) represents the last bottom section. The distance between each optical section is 3 μm. The sections indicate that the fluorescently labelled pDNA molecules are distributed preferentially at the outer peripheral surfaces of the cellulose particles. The fluorescence of the interior of the particles indicates that the pDNA is also distributed on the surfaces of the large pores between the aggregated cellulose particles. The scale bars represent a distance of 20 μm.

FIG. 6 shows an in vitro pDNA release profile from the composition of cellulose carrier particles and pDNA. The pDNA molecules are instantly released from the particles into the buffer solution. More than 70% of the pDNA molecules are released within 5 min.

FIG. 7 shows a macroscopic picture of dissected nasal tissues from mice that received pDNA encoding for the reporter gene, LacZ (pLacZ). Successful transgene expression is indicated by the green-blue colour. Intranasal administration of 25 μg pLacZ alone (a) or in condensed form, using the cationic polymer polyethylenimine (PEI) (c), did not result in transgene expression. In contrast, intranasal administration of a composition of the cellulose carrier particles and pLacZ (25 μg per mouse) (b), resulted in expression of the transgene in the nasal tissue.

FIG. 8 shows histochemical sections of the nasal tissue from mice treated with a composition of cellulose carrier particles and pLacZ, i.e., the same experiment as in FIG. 7b; and of tissue from mice treated with the condensed gene delivery system, i.e. the same experiment as in FIG. 7c. The sections were counter-stained with nuclear fast red. Expression of the reporter gene LacZ is indicated by the blue colour. The epithelium of the nasal tissues from mice that received the composition of cellulose carrier particles and pLacZ expressed the reporter gene (a), while no visible blue colour was found in nasal tissues from mice that received the condensed composition of PEI and pLacZ (b).

FIG. 9 shows that in vivo nasal luciferase expression (pg/mg) 2 days after intranasal administration of 25 ug of the pLuc in rats (three animals per group). As in FIGS. 7 and 8, the pLuc solution and a condensed composition using the cationic polymer PEI were used as controls to the composition of cellulose carrier particles and pLuc. Only the composition of the cellulose carrier particles and pLuc resulted in significant levels of transgene expression, consistent with the results in FIGS. 7 and 8.

FIG. 10 shows in vivo nasal luciferase expression (pg/mg) as a function of time and pDNA formulation following intranasal administration to mice (3-5 animals per group). A solution with pLuc alone and a composition of soluble lactose powder and pLuc were used as controls to the composition of insoluble cellulose carrier particles and pLuc. The dose of pDNA administrated was 25 μg per mouse. Only the composition comprising pLuc associated with the cellulose carrier particles resulted in high levels of transgene expression.

FIG. 11 shows the airway organ distribution of luciferase expression (pg/mg) in mice on day 1 following intranasal administration of 25 μg of the pLuc per mouse (3-5 animals per group). The same compositions as in FIG. 10 was used. Gene expression was only observed in the nasal tissues and no expression was found in the tracheas or in the lungs following the intranasal administration.

FIG. 12 shows the nasal pLuc expression after intranasal administration of two other compositions of alternative insoluble polymeric carrier particles, Kollidon and Ac-Di-Sol, respectively, and pLuc. A composition of cellulose carrier particles and pLuc served as control as did a condensed composition of PEI and pLuc. A composition of naked pLuc in an isotonic solution was also used as a control. Cellulose, Kollidon, Ac-Di-Sol and PEI were formulated as dry powders and tested for pLuc gene expression after intranasal administration to mice (3-5 animals per group) in an amount of 25 μg/mouse. A pLuc expression comparable to that obtained with the cellulose composition was obtained when compositions of any one of the two insoluble non-condensing polymeric carriers were used. In contrast, the condensed formulation of PEI and pLuc, and the naked pLuc solution did not result in transgene expression.

FIG. 13 shows the importance of simultaneous administration of the cellulose carrier particles and pDNA in the composition for the transgene expression in vivo. Administration of pLuc solution to mice at (3-5 animals per group) 5, 15, or 25 min after the administration of cellulose carrier particles did not result in luciferase expression. However, as in the previous examples, the transgene expression was greatly enhanced by using the composition of cellulose carrier particles and pLuc.

FIG. 14 shows the levels of antigen-specific cytokine induction from spleen cells derived from mice that were vaccinated by nasally delivered nucleic acid encoding an antigenic protein. Mice (3-5 animals per group) were nasally inoculated with one microgram of DNA encoding beta-galactosidase on day 0. The DNA was formulated with either CCP, PEI, or NS. Splenocytes were harvested on day 35, cultured in the presence of the beta-galactosidase 876-884 peptide, washed, and restimulated with either the beta-galactosidase 876-884 peptide or a control peptide. Levels of interferon-gamma or interleukin-4 were measured by ELISA.

### DETAILED DESCRIPTION OF THE INVENTION

The present invention is generally related to a composition comprising polymeric carrier particles and nucleic acid, wherein the polymeric carrier particle is comprised of one or more polymers, is essentially insoluble in body fluids and aqueous solutions at a pH less than 8 and wherein further essentially each polymeric particle carries at least one nucleic acid essentially in non-condensed form and associated therewith in such a way that at least part of said nucleic acid is positioned on the surface of the polymeric particle. Suitably, said nucleic acid comprises a coding sequence that will express its function when said nucleic acid is introduced into a host cell.

According to one embodiment of the invention, said nucleic acid is selected from the group consisting of RNA and DNA molecules. These RNA and DNA molecules can be comprised of circular molecules linear molecules or a mixture of both. Suitably, said nucleic acid is comprised of plasmid DNA.

According to one aspect of the present invention, said nucleic acid comprises a coding sequence that encodes a biologically active product, such as a protein, a polypeptide or a peptide having therapeutic, diagnostic, immunogenic, or antigenic activity. An encoded product having antigenic or immunogenic activity suitably comprises an antigenic epitope or determinant that elicits an immune response in a mammal or is a product that acts as a suppressor of an immune response in a mammal.

The present invention is also concerned with compositions as described above wherein said nucleic acid comprises a coding sequence encoding a protein, an enzyme, a polypeptide antigen or a polypeptide hormone or wherein said nucleic acid comprises a nucleotide sequence that functions as an antisense molecule, such as antisense RNA.

A further embodiment of the present invention, is related to compositions wherein the nucleic acid is associated with the polymeric particle in such a way that at least 50 percent by weight of the nucleic acid is positioned within 25 percent by volume of the peripheral parts of the polymeric particle.

Suitably, the polymeric particles of the present invention have a capacity to absorb an aqueous solution in an amount of at least fifty percent in relation to their own weight, and more specifically the polymeric particles are capable of absorbing said amount of aqueous solution within at least 60 seconds and suitably within 10 seconds.

In accordance with the present invention said polymeric particles suitably comprise polymers, e.g. crosslinked polymers, said polymers being selected from the group consisting of natural polymers, modified natural polymers, synthetic polymers, and mixtures thereof.

More specifically, said polymeric particles can comprise natural polymers selected from the group consisting of native cellulose, native starch and mixtures thereof.

Suitably, said native cellulose comprises microcrystalline cellulose.

The present invention is also related to compositions wherein said polymer(s) are comprised of aggregates comprising at least 100 sub-units.

A further aspect of the invention is related to compositions wherein essentially 90% by weight of said polymeric particles have a size smaller than 200 μm, suitably smaller than 100 μm, and preferably smaller that 60 μm. Suitably, essentially 90% by weight of said polymeric particles have a size larger than about 10 μm. Moreover, said polymeric particles suitably have a regular, and specifically a spherical, form.

Another embodiment of the present invention is related to a composition wherein the ratio of positive electric charges and negative electric charges is such that the composition has an essentially net negative charge.

Suitably, the polymeric carrier particles per se are essentially free of groups having a positive electric charge and preferably they are essentially neutral.

In accordance with a further embodiment of the present invention said polymeric carrier particle is essentially insoluble in mucosal fluid.

Another aspect of the present invention is concerned with a composition as described above that further comprises at least one stabilizing agent, such as cryoprotectant.

Suitably said stabilizing agent is selected from sugars and sugar alcohols, such as trehalose, dextrose, sucrose, lactose, sorbitol or mannitol; or amino acids, such as histidine, aspartate, glutamate, lysine, arginine, alanine or glycine.

Illustrative of other stabilizing agents are polyols, such as ethylene glycol, polyethylene glycol or glycerol; ethanol; polysaccharides or polymers, such as inulin, dextran, hydroxyethyl starch, starch, cyclodextrin, heparin, polyvinylpyrrolidone, polyvinylalcohol or surfactants, such as Tween 20, Tween 40, Tween 80, Poloxamer 188, Poloxamer 407, acyl-β-D-maltoside, sodium dodecyl sulphate or cetyltrimethylammonium bromide.

Another embodiment of this invention is concerned with a pharmaceutical preparation comprising a composition as described herein before in a physiologically adminstrable form. Suitably, said preparation is essentially water-free and is administrable to mucosal tissues by oral, buccal, sublingual, rectal, vaginal, pulmonary, or nasal routes, or to submucosal tissues by parenteral routes. Such a preparation can be provided e.g. as a granulate, a tablet, a microtablet, a suppository, an inhalation powder or a nasal powder.

The present invention is also directed to a method for preparing the present composition, said method comprising the steps of:

(a) providing the present polymeric carrier particles disclosed above that are essentially free from water;

(b) providing a solution comprising at least on nucleic acid, as described above, in an aqueous solvent;

(c) contacting the particles from step (a) with the solution from step (b);

(d) drying the product obtained in step (c) to remove the solvent; and

(f) disintegrating the product obtained in step (d) thereby to produce the nucleic acid-carrying polymeric particles of the present invention.

Suitably, the drying in step (d) is achieved by lyophilization.

Moreover, the present method may include a step (e) comprising adding water to the dried product from step (d) in an amount sufficient for reconstitution thereof and subsequently applying speed-vacuum to form a dry cake, after which said dry cake is disintegrated in step (f) by milling.

Suitably, the solution in step (b) further contains at least one stabilizing agent such as a cryoprotectant, e.g. a stabilizing agent as mentioned above.

The present invention is further concerned with a method of administering nucleic acid to a mammal, comprising providing the composition of the present invention; and introducing the composition into the mammal. Suitably, said composition is essentially water-free and is introduced into the mammal by administration to mucosal tissues by oral, buccal, sublingual, rectal, vaginal, pulmonary, or nasal routes. According to a specific embodiment, said composition is essentially water-free and is introduced into the mammal by administration to submucosal tissues by parenteral routes.

A further aspect of the present invention is related to a method of using polymeric carrier particles, which particles are comprised of one or more polymers, are essentially insoluble in body fluids and aqueous solutions at a pH less than 8 and suitably are essentially free of groups having a positive electric charge, to introduce nucleic acid or a polynucleotide into a cell, whereby said nucleic acid or polynucleotide is capable of expressing its function inside said cell.

The present invention is also concerned with use of the composition described above in the manufacture of a medicament for prophylactic or therapeutic treatment of a mammal or in the manufacture of a diagnostic agent for in vivo or in vitro diagnostic methods, and specifically in the manufacture of a medicament for use in gene therapy, antisense therapy or genetic vaccination for prohylactic or therapeutic treatment of malignancies, autoimmune diseases, inherited disorders, pathogenic infections and other pathological diseases.

Further aspects of this invention are concerned with methods of inducing an immune response against a protein in a mammal or delivering a protein into the systemic circulation of an animal, said methods comprising mucosal or nasal delivery of DNA using a composition as described herein before.

### EXAMPLES

### Example 1

Preparation and Characterization of Polymeric Carrier Particles

The cellulose carrier particles were prepared according to a method described earlier (Orlando et al, U.S. Pat. No. 3,146,168, 1964) with modification. Briefly, Avicel microcrystalline cellulose (NFEP, FMC, Ireland) was hydrolyzed in distilled water (dH₂O) at 50° C. and a pH of 1 for 6 days, followed by washing with dH₂O (50° C.) until the pH increased to 5. The cellulose was then vacuum-dried to a hard paste. The cellulose paste was milled by a mortar grinder (Retsch KM1, Germany) until the thread shaped cellulose disappeared as observed under a light microscope. The grinded cellulose samples were resuspended in dH₂O at 50% (w/v) consistency and stirred by a mixer (Silverson Machines Ltd., UK), followed by spray drying (A/S Niro Automister, Copenhagen, Denmark).

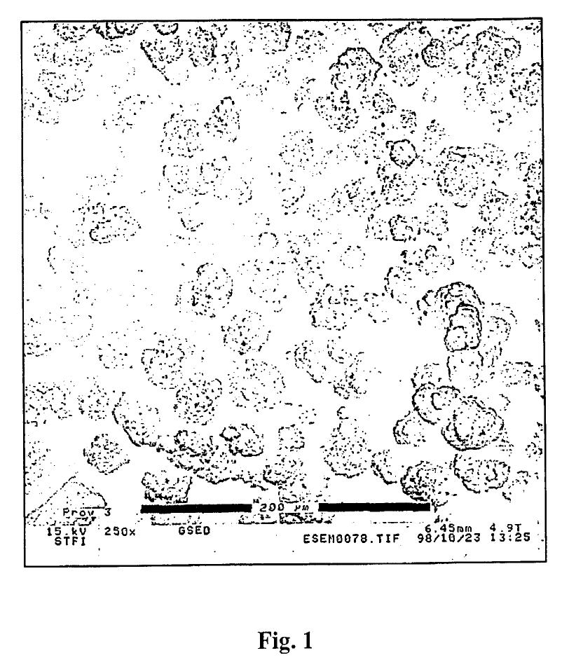

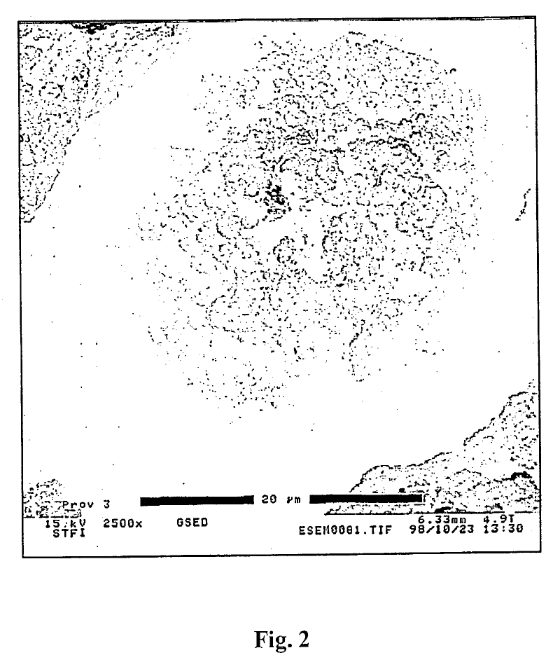

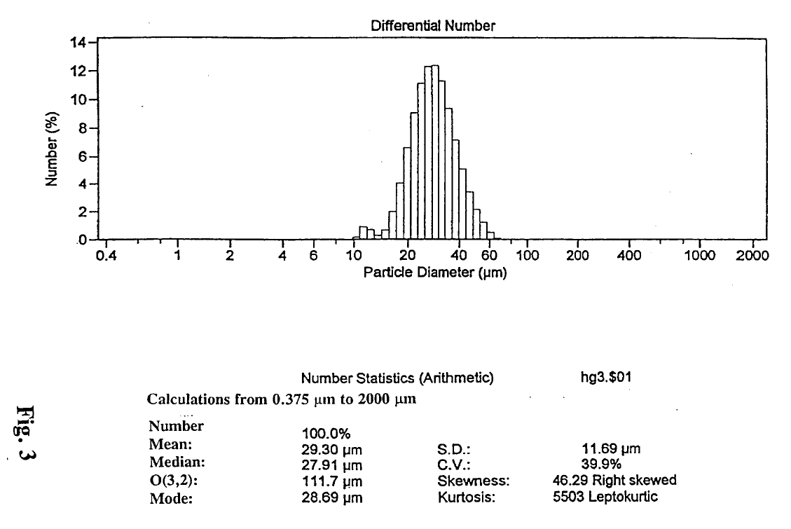

The spray-dried product was further processed. Firstly, the cellulose carrier particles were separated into small (<10 μm) and large (>10 μm) size groups by an air elutriation apparatus (Alpine 100 MZR, Alpine AG, Germany) followed by washing with ddH₂O and ethanol to produce free flowing powders of the cellulose carrier particles. A sample of the cellulose carrier particles was examined by scanning electron microscopy as shown in FIGS. 1 and 2. The size distribution of the cellulose carrier particle aggregates was analyzed by a laser particle size analyzer (Coulter LS 230, Coulter Corporation, FL, USA) as shown in FIG. 3. The size distribution was found to follow a normal-log size distribution.

### Example 2

Formulation and Characterization of a Composition Comprised of of Polymeric (Cellulose) Particles and pDNA

Firefly luciferase plasmid DNA (pLuc, supercoiled form greater than 95.8%) and psoraten fluorescence labeled pDNA of GMP grade were kindly provided by GeneMedicine Inc., TX, USA. Formulation stabilizing agents cellobiose and alpha-lactose or D(+)-galactose were purchased from Pfanstiehl laboratories Inc., IL, USA, and Sigma, St. Louise, USA, respectively. The pDNA-loading dose was 60 μg per mg of the polymeric carrier particles and the stabilizing agent, unless otherwise is indicated. The w/w ratio of a stabilizing agent to the polymeric carrier particles was ⅓. First, one of the stabilizing agents was added into the pDNA solution followed by mixing (Vortex Heidolph REAX 2000, KEBO Lab, Spånga, Sweden). Then, the polymeric carrier particles were added into the mixture. After mixing, the resulting suspension was frozen in liquid nitrogen for 5 min and lyophilized at −40° C. and 400 Pa (Edwards Pirani 501, Germany) overnight. The freeze-dried powder was reconstituted in distilled deionized water (ddH₂O) of a sufficient volume to wet the freeze-dried cellulose particles. Then, the wetted particles were dried to form a dry cake in a speed-vacuum centrifuge (e.g., SpeedVac Plus SC 110A, Savant Instruments, NY, USA) at room temperature. Finally, the dry cake was milled into a fine powder.

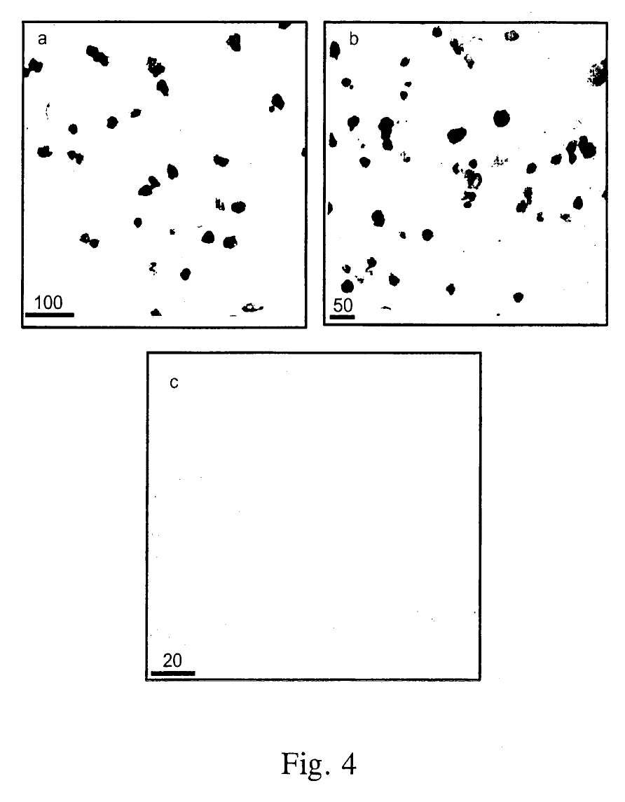

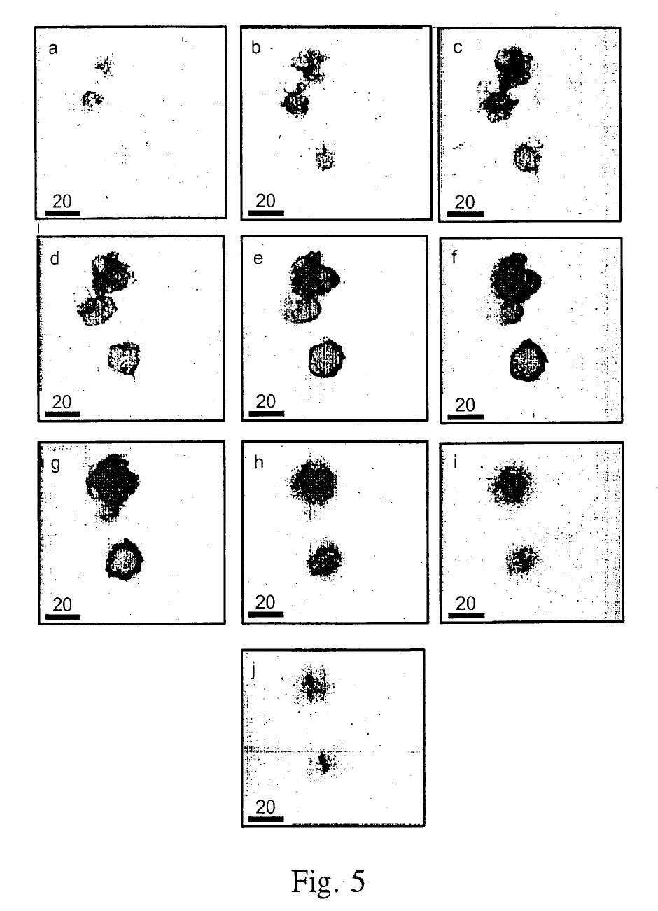

A rough assessment of the size distribution of the composition of cellulose carrier particles and pDNA was performed by a confocal laser-scanning microscopy (Leica TC4D Confocal Scanner, Leica Laser Technik GmbH, Heidelberg, Germany). Two fields were randomly chosen (FIGS. 4a and 4b) and the length and width of each particle were measured. A total of 380 particles were counted, giving a mean diameter of about 20 μm and 24.7 μm for width and length, respectively (FIG. 4). Thus, the composition of cellulose carrier particles and pDNA had a relatively homogenous size distribution comparable to that of the cellulose particles (FIG. 3). FIG. 4c was a control generated by scanning cellulose carrier particles free of the fluorescence labelled pDNA, showing that all the green colour was contributed by the pDNA rather than the cellulose. In order to examine the location of pDNA in the cellulose carrier particles, optical sections of the composition of cellulose carrier particles and pDNA were performed by a scanning confocal microscope (FIG. 5). The results indicate that the pDNA was attached to outer surfaces and inner, i.e. pore, surfaces of the particles.

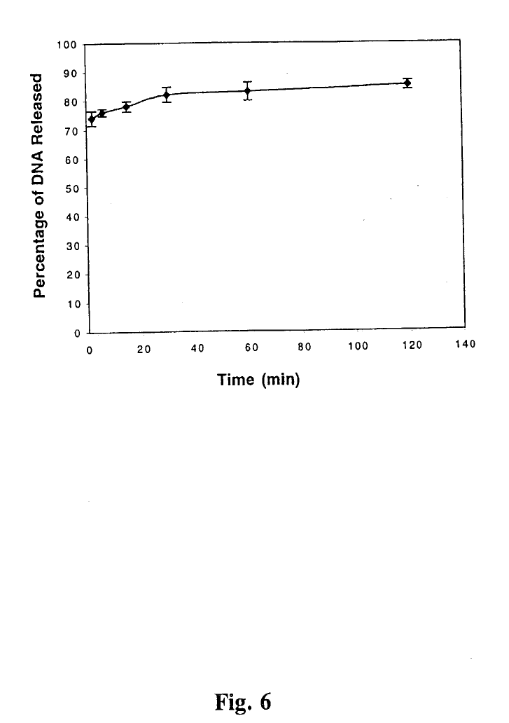

The release of pDNA from the composition of cellulose carrier particles and pDNA was investigated in vitro at room temperature. The pDNA-loading dose was 12.5 μg per mg of the cellulose carrier particles in this study. Aliquots of the composition of cellulose carrier particles and pDNA were added to 500 μl of PBS (pH 7.4, 54 mM, without Ca²⁺/Mg²⁺). At various time intervals, the pDNA released into the supernatant was quantified by an UV spectrometer (Unicom Ltd., Cambridge, UK) at a wave length of 260 nm. Supernatant from a corresponding suspension of cellulose carrier particles free of pDNA served as the reference. Results showed an instant release, more than 70% of the pDNA molecules being released within the first 5 min (FIG. 6).

### Example 3

Qualitative Transgene Expression After Intranasal Administration of a Composition Comprised of Polymeric (Cellulose) Carrier Particles and pDNA

In order to assess the effectiveness of various pDNA compositions on gene transfection and expression in vivo, a plasmid containing the LacZ reporter gene (pLacZ, (Berglund et al. 1998)) was used in isotonic solution alone and formulated into compositions of cellulose carrier particles as described in Example 2. First, one of the stabilizing agents was added into the pLacZ solution followed by mixing (Vortex Heidolph REAX 2000, KEBO Lab, Spånga, Sweden). Then, the cellulose carrier particles were added into the mixture. After mixing, the resulting suspension was frozen in liquid nitrogen for 5 min and lyophilized at −40° C. and 400 Pa (Edwards Pirani 501, Germany) overnight. The freeze-dried powder was reconstituted in distilled deionized water (ddH₂O) of a sufficient volume to wet the freeze-dried cellulose particles. Then, the wetted particles were dried to form a dry cake in a speed-vacuum centrifuge (e.g., SpeedVac Plus SC110A, Savant Instruments, NY, USA) at room temperature. Finally, the dry cake was milled into a fine powder. In addition, pLacZ was formulated with PEI at a previously optimized condition of PEI (25 kD) : pDNA at 5:1 (Boussif et al. 1995; Köping-Höggård et al. 1998).

Mice (female 7-9 weeks old Balb/c, 3-5 animals per group, Charles River, Uppsala, Sweden) were anaesthetized with Ketamin and Xylazine (40% v/v Katalar and 10% v/v Xylazine in ddH₂O) at a dose of 50 μl per log-body weight of the animal. An amount of 25 μg of the pLacZ associated with either cellulose or PEI carrier particles or an amount of 20 μl of the pLacZ isotonic solution was delivered into one of the nasal cavities of the animal via syringes or pipettes. About 10 min after euthanization and perfusion with PBS, the nasal tissues of each group were firstly fixed in 3% paraformaldehyde for 3 hours at 4° C., followed by incubation in X-gal solution at 37° C. overnight (Weiss et al. 1997). The nasal tissues were then post-fixed in the same fixation solution at 4° C. overnight, followed by decalcification, standard tissue sectioning and counter-staining with nuclear fast red.

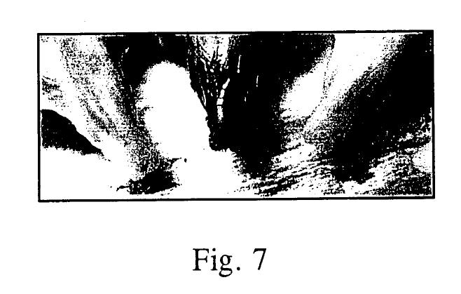

FIG. 7 shows the significant differences in LacZ gene transfection and expression from mice administrated with various compositions of the pLacZ. The level of the LacZ gene expression is indicated by the blue-green color of the nasal tissue. Surprisingly, only the composition of cellulose carrier particles and pLacZ showed a significant level of gene expression (FIG. 7b), whereas the other compositions (PEI and pLacZ and pLacZ alone in solution, respectively) did not result in gene expression (FIGS. 7a and 7c). This result indicates that the composition of cellulose carrier particles and pLacZ can enhance the gene transfection in vivo.

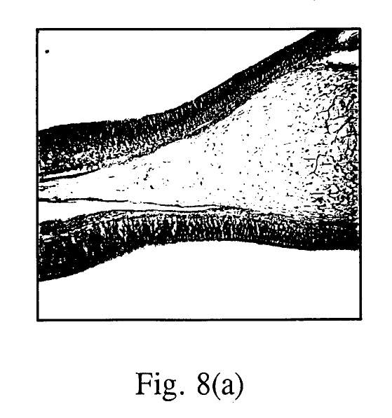

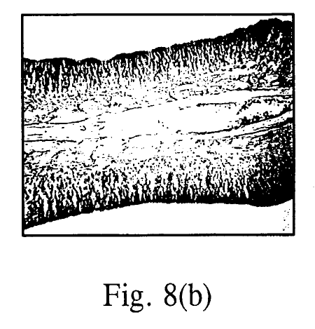

FIG. 8 shows histochemical sections of the nasal tissue from mice in the same experiment as in FIG. 7b; and of a control, i.e the same as in FIG. 7c. The sections were counter-stained with nuclear fast red. Expression of the reporter gene LacZ is indicated by the blue color. The stained sections show that the epithelium of the nasal tissues was transfected with the composition of cellulose carrier particles and pLacZ (FIG. 8a), while no visible blue color was found in nasal tissues from mice that received the condensed composition of PEI and pLacZ (FIG. 8b).

### Example 4

Quantitative Transgene Expression After Intranasal Administration of Compositions Comprised of Polymeric Carrier Particles and pDNA

Gene expression was also studied after transfection with compositions containing a plasmid encoding the enzyme luciferase. A plasmid containing the luciferase reporter gene (pLuc) was formulated in isotonic solution alone. Three different compositions of insoluble polymer particles of either cellulose or Kollidon® (insoluble cross-linked PVP) or Ac-Di-Sol® (modified starches and cellulose derivatives/sodium carboxymethylcellulose) carrier particles and pLuc and two compositions of soluble carrier particles comprised of lactose and pLuc or of PEI and pLuc were formulated as described in Example 3.

Rats (male, 5 weeks old, Sprague-Dawley, Charles River, Uppsala, Sweden) or mice (female, 7-9 weeks old Balb/c, 3-5 animals per group, Charles River, Uppsala, Sweden) were administered with the various compositions at an amount of 25 μg of the pLuc per animal as described in Example 3. At indicated time points after administration, the treated animals and untreated control animals were sacrificed by carbon dioxide and the airway tissues of nose, trachea and lungs were surgically removed, quickly frozen in liquid nitrogen and stored at −80° C. until analysis.

In a cold room, the tissue samples were homogenized (Homogenize, Biospec. Products, Inc., OK, USA) in ice-cold lysis buffer (Promega, WI, USA) containing a protease inhibitor cocktail (Boehringer Mannheim AB, Bromma, Sweden). Then the samples were centrifuged (Centrifuge 5403, Eppendorf-Nethelar-Hinze GmbH, Hamburg, Germany) at 4° C. and 15,000 rpm for 8 min. An amount of 50 μl of the clear supernatant from each test tube was mixed with 50 μl of luciferase reagent (Promega Corporation, WI, USA) and analyzed by a luminometer (Mediators PhL, Vienna, Austria) with an integration time of 8 s. In order to quantify the luciferase expression, a standard curve of luciferase (Sigma, St. Louise, Mo., USA) was prepared for each experiment by adding defined amounts of the luciferase standard to the supernatants of homogenized tissues from the untreated control animals. The total protein content in each sample was analyzed by the BCA assay (Micro BCA, Pierce, Ill., USA) and quantified using BSA (bovine serum albumin) (Sigma, St. Louise, Mo., USA) as a reference protein. The absorbance was measured at 540 nm on a microplate reader (FL5000, Bio-Tek Instruments, Spånga, Sweden).

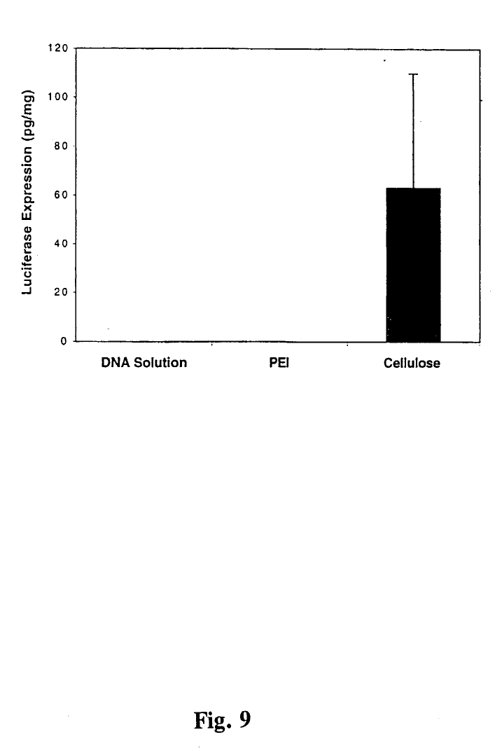

FIG. 9 shows the gene transfection activity of various compositions of carriers and pLuc in rats. A composition of PEI (Aldrich Sweden, Stockholm, Sweden) carrier and pLuc prepared as described in Example 3 was applied as a positive control. Surprisingly, a significant amount of luciferase was only expressed in the nasal tissues of rats administered with the composition of cellulose carrier particles and pDNA, whereas there was no detectable level of luciferase expression in rats administered with the other two compositions.

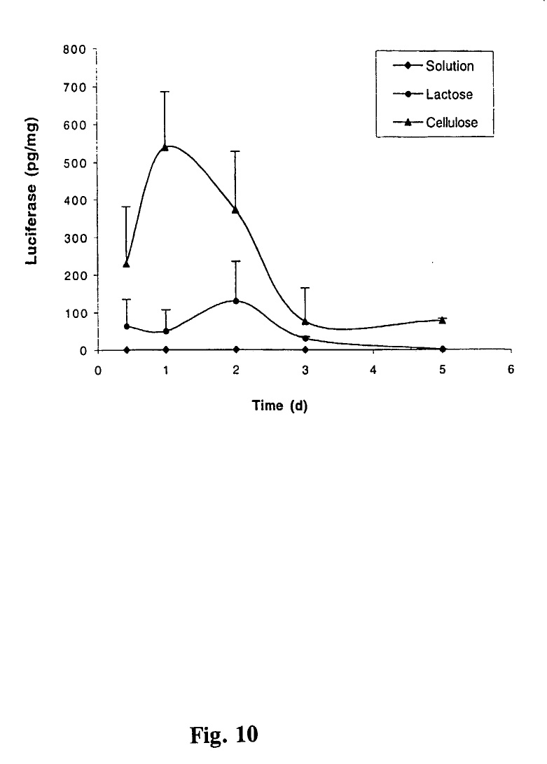

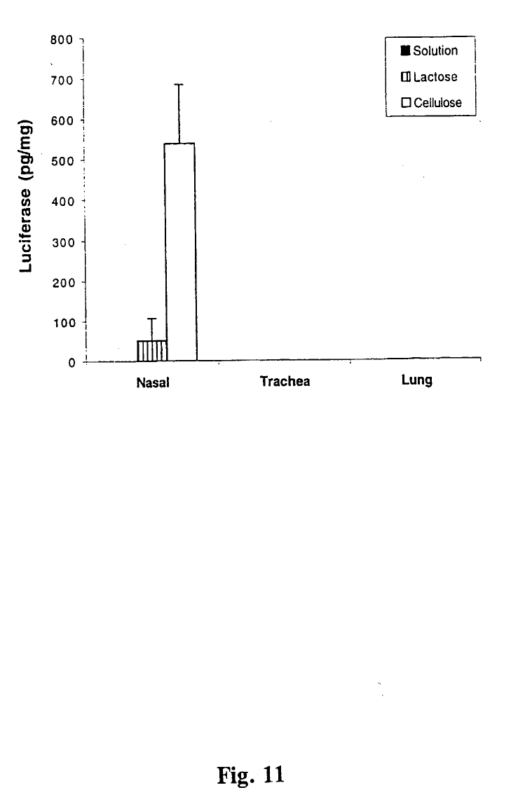

FIG. 10 shows the time dependent luciferase gene expression after intranasal administration of pLuc solution or the compositions of pLuc and cellulose carriers or PEI, respectively. Expression of pLuc was assessed kinetically in the tissues of nasal, trachea and lung in mice (male, 7-10 weeks old, Balb/c). Gene expression from the composition of cellulose carrier particles and pLuc showed a fast onset as a significant amount of luciferase activity was detected already after 10 hours and luciferase expression peaked on day 1. A much lower luciferase expression was obtained from the composition of comprised of instantly soluble lactose particles and pLuc, while no expression was detected from the pLuc solution (FIG. 11). Interestingly, no gene expression was observed in the trachea or lungs, suggesting that the gene expression was confined to an area in the vicinity of the administration site (FIG. 11).

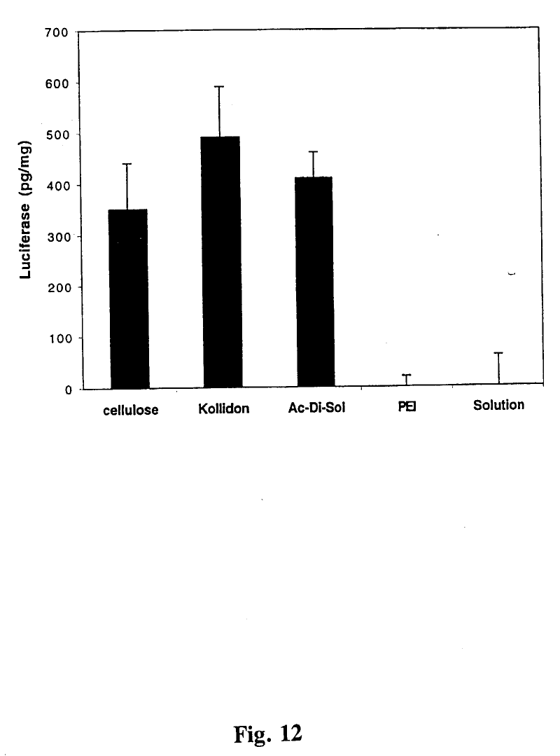

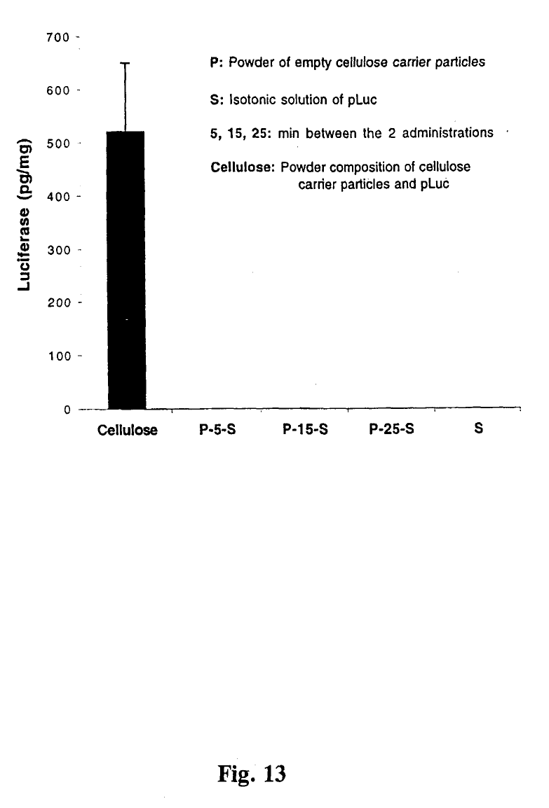

FIG. 12 shows pLuc expression after intranasal administration of two compositions of pLuc and two other insoluble, polymeric particles that are essentially free of groups having a positive electric and have the capacity to swell in water, i.e. Kollidon® and Ac-Di-Sol®. Control formulations were composed of PEI and pLuc or pLuc solution alone. The results show a comparable level of gene expression from the various compositions of pLuc and polymeric carriers with similar physic-chemical properties, whereas there was no gene expression from controls of the condensed pLuc (the composition of pLuc and PEI) or pLuc in solution. Interestingly, as shown in FIG. 13, the enhanced gene transfection and luciferace expression achieved with the composition of cellulose carrier particles and pLuc could not be reproduced by a separate administration of the cellulose carrier particles followed by the pLuc solutions, suggesting the importance of simultaneous administration of the cellulose carrier particles and pDNA in the composition.

### Example 5

Use of Nasal Delivery to Induce an Immune Response in an Animal

To address whether nasal delivery of nucleic acid could be used to induce an immune response against an antigen encoded by the nasally delivered nucleic acid groups of three to five mice were immunized intranasally on either day 0 or day 0 and day 14 with a plasmid encoding E. coli beta-galactosidase formulated with either the preferred cellulose carrier particle formulation of the invention (CCP), with a polyethyleneimine-based formulation (PEI), or naked DNA (‘naked solution;’ NS). Doses of 1, 25, or 75 micrograms per mouse were used. On day 35, spleen and serum samples from each animal were collected and pooled. Spleen cells were cultured under standard conditions in the presence of 1 microgram/ml beta-galactosidase₈₇₆₋₈₈₄ peptide, followed by washing and restimulation with either beta-galactosidase₈₇₆₋₈₈₄ peptide or a control peptide for 18-22 hours. Supernatants of restimulated cultures were collected and assayed for release of IFN-gamma and IL-4 using ELISA kits (Pharmingen, San Diego USA). From serum samples, IgG₁, IgG₂ₐ, IgG_{2b}, IgG₃, and IgA anti-beta-galactosidase antibodies were also analyzed by ELISA. Feces were also collected and analyzed for IgA specific to beta-galactosidase.

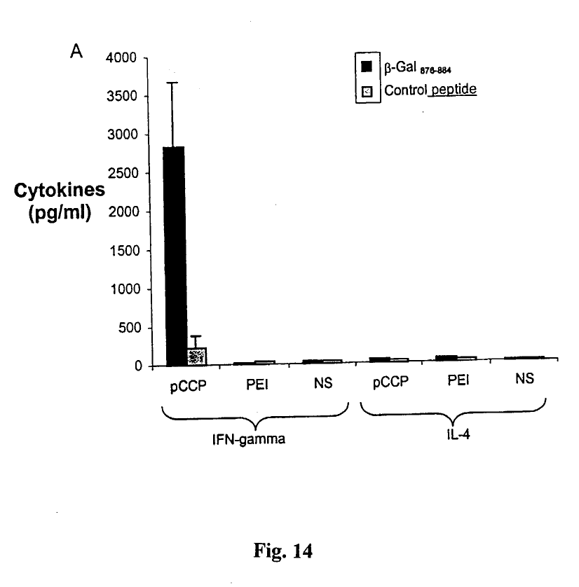

A single intranasal administration of one microgram of DNA encoding beta-galactosidase formulated with CCP resulted in a strong antigen-specific cellular immune response. FIG. 14 shows typical results. The response was demonstrated by induction of interferon-gamma but not interleukin-4, indicating a Thl response had occurred. No detectable response was observed with the other formulations.

An antibody response was also observed. Beta-galactosidase-specific antibodies of the IgGl subclass were detected in animals treated with 1, 25, or 75 micrograms per mouse.

### Example 6

Use of Nasal Delivery of DNA to Deliver a Therapeutic Protein to an Animal

The methods described above are used to indirectly deliver therapeutic proteins to mammals, such as a mouse, rabbit, monkey, or human. For example, an expression plasmid encoding a secreted protein is formulated with a cellulose carrier particle and administered to an animal. Cells of the nasal epithelium take up the expression plasmid, transcribe the gene encoding the secreted protein, translate the resulting mRNA, and secrete the protein. The secreted protein is detected in the systemic circulation of the animal.

In summary, the examples above show that according to the present invention epithelial cells of a mucosal tissue can quite unexpectedly be efficiently transfected with the present composition comprising essentially water-insoluble polymeric carrier particles that are essentially without positive charges and wherein essentially non-condensed nucleic acid, e.g. pDNA, is localized on the surface of the carrier particles. This simple formulation is cheap and easy to prepare in a dry form that will be easy to store. Therefore, it is suitable for large-scale production. It can be applied in various forms in human gene/antisense therapy and genetic vaccination. For example, it can be formulated as a nasal spray or as an inhalation powder for vaccination purposes or for the treatment of diseases localized to the airway epithelium, such as cystic fibrosis. It is obvious for a person skilled in the art that the dry particle powders can be administered also to other mucosal tissues. According to a further embodiment of the invention that is easily comprehended by a person skilled in the art, the composition can be compressed into tablets or filled into capsules for oral administration. It is also obvious for a person skilled in the art that other nucleic acid drugs, such as oligo-nucleotides can be delivered by the same principle. Illustrative therapeutic, prophylactic and diagnostic applications of the new compositions described here include, but are in not limited to, those embodied in the fields of gene therapy, anti-sense therapy and genetic vaccination. Thus, while a number of suitable embodiments of the invention have been described, these are intended to illustrate but not to limit the present invention. Other aspects, advantages, and modifications are within the scope of the following claims.

All patents and publications mentioned in the specification are herein incorporated by reference to the same extent as if each individual publication was specifically and individually indicated to be incorporated by reference.

### REFERENCES

(1) Ando et al., “PLGA microspheres containing plasmid DNA: preservation of supercoiled DNA via cryopreparation and carbohydrate stabilization [In Process Citation],” J Pharm Sci, 88:126-30, 1999.

(2) Armstrong et al., “A polynucleotide herpes virus vaccine,” WO 98/14586, USA, Merck & Co., Inc., 1997.

(3) Barry et al., “Needle-free injection of formulated nucleic acid molecules,” PCT. US, Baylor College of Medicine, 1999.

(4) Barthel et al., “Gene transfer optimization with lipospermine-coated DNA,” DNA Cell Biol, 12:553-60, 1993.

(5) Berglund et al., “Enhancing immune responses using suicidal DNA vaccines, “Nat Biotechnol, 16:562-5, 1998.

(6) Bot et al., “Immunization of infants,” WO 98/22145, USA, Mount Sinai School of Medicine of the City Uuniversity of New York, 1998.

(7) Boussif et al., “A versatile vector for gene and oligonucleotide transfer into cells in culture and in vivo: polyethylenimine,” Proc Natl Acad Sci USA, 92:7297-301, 1995.

(8) Capan et al., “Preparation and characterization of poly (D,L-lactide-co-glycolide) microspheres for controlled release of poly(L-lysine) complexed plasmid DNA,” Pharm Res, 16:509-13, 1999.

(9) Deshpande et al., “Target specific optimization of cationic lipid-based systems for pulmonary gene therapy,” Pharm Res, 15:1340-7, 1998.

(10) Fasbender et al., “Optimization of cationic lipid-mediated gene transfer to airway epithelia,” Am J Physiol, 269:L45-51, 1995.

(11) Felgner et al., “Lipofection: a highly efficient, lipid-mediated DNA-transfection procedure,” Proc Natl Acad Sci USA, 84:7413-7, 1987.

(12) Freimark et al., “Cationic lipids enhance cytokine and cell influx levels in the lung following administration of plasmid: cationic lipid complexes,” J Immunol, 160:4580-6, 1998.

(13) Gao et al., “Cationic liposome-mediated gene transfer,” Gene Ther, 2:710-22, 1995.

(14) Guillaume-Gable et al., “Cationic phosphonolipids as nonviral gene transfer agents in the lungs of mice,” Hum Gene Ther, 9:2309-19, 1998.

(15) Hedley et al., “Microspheres containing plasmid-encoded antigens elicit cytotoxic T-cell responses,” Nat Med, 4:365-8, 1998.

(16) Hedley et al., “Microparticles for delivery of nucleic acid,” 5,783,567, USA, Pangaea Pharmaceuticals, Inc., 1998.

(17) Jiang et al., “Efficiency of cationic lipid-mediated transfection of polarized and differentiated airway epithelial cells in vitro and in vivo,” Hum Gene Ther, 9:1531-42, 1998.

(18) Jones et al., “Poly(DL-lactide-co-glycolide)-encapsulated plasmid DNA elicits systemic and mucosal antibody responses to encoded protein after oral administration,” Vaccine, 15:814-7, 1997.

(19) Jones et al., “Oral delivery of poly(lactide-co-glycolide) encapsulated vaccines,” Behring Inst Mitt, 220-8, 1997.

(20) Kabanov et al., “DNA complexes with polycations for the delivery of genetic material into cells,” Bioconjug Chem, 6:7-20, 1995.

(21) Köping-Höggård et al., “Chitosan-pDNA polyplex: a new efficient and biodegradable gene delivery system in vitro,” PharmSci, 1:S-285, 1998.

(22) Köping-Höggård et al., “Chitosan-pDNA polyplex: in vivo gene expression after tracheal, nasal and oral administration to mice,” PharmSci, 1:S-278, 1998.

(23) Lee et al., “Lipidic vector systems for gene transfer,” Crit Rev Ther Drug Carrier Syst, 14:173-206, 1997.

(24) Lewis et al., “Development of a sustained-release biodegradable polymer delivery system for site-specific delivery of oligonucleotides: characterization of P(LA-GA) copolymer microspheres in vitro,” J Drug Target, 5:291-302, 1998.

(25) Meyer et al., “Intratracheal gene delivery to the mouse airway: characterization of plasmid DNA expression and pharmacokinetics,” Gene Ther, 2:450-60, 1995.

(26) Miglierina “Some applications of microspheres in flow cytometry,” Biol Cell, 58:127-30, 1986.

(27) Mumper et al., “Polyvinyl derivatives as novel interactive polymers for controlled gene delivery to muscle,” Pharm Res, 13:701-9, 1996.

(28) Mumper et al., “Protective interactive noncondensing (PINC) polymers for enhanced plasmid distribution and expression in rat skeletal muscle,” J Controlled Release, 52:191-203, 1998.

(29) Pillai et al., “Ultrasonic nebulization of cationic lipid-based gene delivery systems for airway administration [In Process Citation],” Pharm Res, 15:1743-7, 1998.

(30) Saeki et al., “Development and characterization of cationic liposomes conjugated with HVJ (Sendai virus): reciprocal effect of cationic lipid for in vitro and in vivo gene transfer,” Hum Gene Ther, 8:2133-41, 1997.

(31) Schwartz et al., “Gene transfer by naked DNA into adult mouse brain,” Gene Ther, 3:405-11, 1996.

(32) Schwartz et al., “Lipospermine-based gene transfer into the newborn mouse brain is optimized by a low lipospermine/DNA charge ratio,” Hum Gene Ther, 6:1515-24, 1995.

(33) Shea et al., “DNA delivery from polymer matrices for tissue engineering [see comments],” Nat Biotechnol, 17:551-4, 1999.

(34) Sternberg et al., “Ultrastructural characterization of cationic liposome-DNA complexes showing enhanced stability in serum and high transfection activity in vivo,” Biochim Biophys Acta, 1375:23-35, 1998.

(35) Wang et al., “Encapsulation of plasmid DNA in biodegradable poly(D, L-lactic-co-glycolic acid) microspheres as a novel approach for immunogene delivery,” J Controlled Release, 57:9-18, 1999.

(36) Weiss et al., “In situ histochemical detection of beta-galactosidase activity in lung: assessment of X-Gal reagent in distinguishing lacZ gene expression and endogenous beta-galactosidase activity,” Hum Gene Ther, 8:1545-54, 1997.

(37) Wolff et al., “Direct gene transfer into mouse muscle in vivo,” Science, 247:1465-8, 1990.

(38) Zabner et al., “Comparison of DNA-lipid complexes and DNA alone for gene transfer to cystic fibrosis airway epithelia in vivo,” J Clin Invest, 100:1529-37, 1997.

1. A composition comprising polymeric carrier particles and nucleic acid, wherein the polymeric carrier particle is comprised of one or more polymers, is essentially insoluble in body fluids and aqueous solutions at a pH less than 8; and wherein further essentially each polymeric particle carries at least one nucleic acid essentially in a non-condensed form and associated therewith in such a way that at least part of said nucleic acid is positioned on the surface of the polymeric particle.

2. The composition of claim 1, wherein said polymeric carrier particle is essentially free of groups having a positive electric charge.

3. The composition of claim 1, wherein said polymeric carrier particle comprises a coding sequence that will express its function when said nucleic acid is introduced into a host cell.

4. The composition of claim 1, wherein said nucleic acid is selected from the group consisting of RNA and DNA molecules.

5. The composition of claim 4, wherein said DNA is comprised of plasmid DNA.

6. The composition of any preceding claim, wherein said coding sequence encodes a biologically active product.

7. The composition of claim 6, wherein said encoded product is a protein, a polypeptide or a peptide having therapeutic, diagnostic, immunogenic, or antigenic activity.

8. The composition of claim 7, wherein said product having antigenic or immunogenic activity is a product that comprises an antigenic epitope or determinant that elicits an immune response in a mammal or is a product that acts as a suppressor of an immune response in a mammal.

9. The composition of claim 6, wherein said biologically active product is a protein, an enzyme, a polypeptide antigen or a polypeptide hormone.

10. The composition of claim 1, wherein said nucleic acid comprises a nucleotide sequence that functions as an antisense molecule, such as antisense RNA.

11. The composition of any preceding claim, wherein the nucleic acid is associated with the polymeric particle in such a way that at least 50 percent by weight of the nucleic acid is positioned within 25 percent by volume of the peripheral parts of the polymeric particle.

12. The composition of any preceding claim, wherein the polymeric particles have a capacity to absorb an aqueous solution in an amount of at least fifty percent in relation to their own weight.

13. The composition of claim 12, wherein the polymeric particles are capable of absorbing said amount of aqueous solution within at least 60 seconds and suitably within 10 seconds.

14. The composition of any preceding claim, wherein the polymeric particles comprise polymers selected from the group consisting of natural polymers, modified natural polymers, synthetic polymers, and mixtures thereof.

15. The composition of claim 14, wherein said polymeric particles comprise natural polymers selected from the group consisting of native cellulose, native starch and mixtures thereof.

16. The composition of claim 15, wherein said native cellulose comprises microcrystalline cellulose.

17. The composition of claim 14, wherein said polymers are comprised of cross-linked polymers.

18. The composition of any preceding claim, wherein said polymer(s) are comprised of aggregates comprising at least 100 sub-units.

19. The composition of any preceding claim, wherein essentially 90% by weight of said polymeric particles have a size smaller than 200 μm, suitably smaller than 100 μm, and preferably smaller than 60 μm.

20. The composition of claim 1, wherein essentially 90% by weight of said polymeric particles have a size larger than about 10 μm.

21. The composition of claim 18 or 19, wherein said polymeric particles have a regular, and suitably a spherical, form.

22. The composition of any preceding claim, wherein the ratio of positive electric charges and negative electric charges is such that the composition has an essentially net negative charge.

23. The composition of claim 22, wherein the polymeric carrier particles per se are essentially neutral.

24. The composition of any preceding claim, wherein the polymeric carrier particle is essentially insoluble in mucosal fluid.

25. The composition of any preceding claim, wherein said composition further comprises at least one stabilizing agent such as a cryoprotectant.

26. The composition of claim 25, wherein said stabilizing agent is selected from sugars and sugar alcohols, such as trehalose, dextrose, sucrose, lactose, sorbitol or mannitol; or amino acids, such as histidine, aspartate, glutamate, lysine, arginine, alanine or glycine.

27. The composition of claim 25, wherein said stabilizing agent is selected from polyols, such as ethylene glycol, polyethylene glycol or glycerol; ethanol; polysaccharides or polymers, such as inulin, dextran, hydroxyethyl starch, starch, cyclodextrin, heparin, polyvinylpyrrolidone, polyvinylalcohol or surfactants, such as Tween 20, Tween 40, Tween 80, Poloxamer 188, Poloxamer 407, acyl-β-D-maltoside, sodium dodecyl sulphate or cetyltrimethylammonium bromide.

28. A pharmaceutical preparation comprising the composition of any preceding claim in a physiologically administrable form.

29. The preparation of claim 28, wherein the preparation is essentially water-free and is administrable to mucosal tissues by oral, buccal, sublingual, rectal, vaginal, pulmonary, or nasal routes, or to submucosal tissues by parenteral routes.

30. The preparation of claim 29, which is a granulate, a tablet, a microtablet, a suppository, an inhalation powder or a nasal powder.

31. A method for preparing the composition of claim 1, comprising the steps of:
(a) providing polymeric carrier particles of claim 1 that are essentially free from water;
(b) providing a solution comprising at least one nucleic acid as defined in claim 1 in an aqueous solvent;
(c) contacting the particles from step (a) with the solution from step (b);
(d) drying the product obtained in step (c) to remove the solvent; and
(f) desintegrating the product obtained in step (d) thereby to produce the nucleic acid-carrying polymeric particles of claim 1.

32. The method of claim 31, wherein the drying in step (d) is achieved by lyophilization.

33. The method of claim 31, which method further includes a step (e) comprising adding water to the dried product from step (d) in an amount sufficient for reconstitution thereof and subsequently applying speed-vacuum to form a dry cake, after which said dry cake is desintegrated in step (f) by milling.

34. The method of claim 31, 32, or 33, wherein the solution in step (b) further contains at least one stabilizing agent such as a cryoprotectant.

35. The method of claim 34, wherein said stabilizing agent is selected from sugars and sugar alcohols, such as trehalose, dextrose, sucrose, lactose, sorbitol or mannitol; or amino acids, such as histidine, aspartate, glutamate, lysine, arginine, alanine or glycine.

36. The method of claim 34, wherein said stabilizing agent is selected from polyols, such as ethylene glycol, polyethylene glycol or glycerol; ethanol; polysaccharides or polymers, such as inulin, dextran, hydroxyethyl starch, starch, cyclodextrin, heparin, polyvinylpyrrolidone, polyvinylalcohol and surfactants, such as Tween 20, Tween 40, Tween 80, Poloxamer 188, Poloxamer 407, acyl-β-D-maltoside, sodium dodecyl sulphate or cetyltrimethylammonium bromide, or a salt, such as sodium chloride.

37. A method of administering nucleic acid to a mammal, comprising providing the composition of claim 1; and introducing the composition into the mammal.

38. The method of claim 37, wherein the composition is essentially water-free and is introduced into the mammal by administration to mucosal tissues by oral, buccal, sublingual, rectal, vaginal, pulmonary, or nasal routes.

39. The method of claim 37, wherein the composition is essentially water-free and is introduced into the mammal by administration to submucosal tissues by parenteral routes.

40. A method of using polymeric carrier particles, which particles are comprised of one or more polymers, and are essentially insoluble in body fluids and aqueous solutions at a pH less than 8, to introduce nucleic acid or a polynucleotide into a cell, whereby said nucleic acid or polynucleotide is capable of expressing its function inside said cell.

41. The use of the composition of claim 1 in the manufacture of a medicament for prophylactic or therapeutic treatment of a mammal or in the manufacture of a diagnostic agent for in vivo or in vitro diagnostic methods.

42. The use of the composition of claim 1 in the manufacture of a medicament for use in gene therapy, antisense therapy or genetic vaccination for prophylactic or therapeutic treatment of malignancies, autoimmune diseases, inherited disorders, pathogenic infections and other pathological diseases.

43. A method of inducing an immune response against a protein in a mammal, said method comprising the mucosal delivery of DNA using the composition of claim 1.

44. A method of delivering a protein into the systemic circulation of an animal, said method comprising the mucosal delivery of DNA using the composition of claim 1.

45. A method of inducing an immune response against a protein in a mammal, said method comprising the nasal delivery of DNA using the composition of claim 1.

46. A method of delivering a protein into the systemic circulation of an animal, said method comprising the nasal delivery of DNA using the composition of claim 1.

47. The method of claim 40, wherein said carrier particles are essentially free of groups having a positive electric charge.
